(19) United States
(12) Patent Application Publication
(10) Pub. No.: US 2002/0131888 A1
(43) Pub. Date: Sep. 19, 2002

(54) Apparatus and method for diffusion of solutions including a method of manufacturing

(75) Inventors: Franco Zobele, Trento (IT); Fabio Marchetti, Povo (IT)

Correspondence Address: Russell Carter Gache, Esq., Sirote & Permutt, P.C., P.O. Box 55727, Birmingham, AL, 35255-5727, US

(73) Assignee: Zobele Holding SpA

(21) Appl. No.: 10/098,603

(22) Filed: Mar. 15, 2002

(30) Foreign Application Priority Data
Mar. 16, 2001 (IT) MI2001A 000559

(51) Int. Cl. A61L 9/00; B32B 5/02; A62B 7/08; B32B 27/04; B32B 27/12; A61L 2/00; B01J 7/00

(52) U.S. Cl. 422/5; 422/123; 422/125; 422/292; 422/305

(57) ABSTRACT

A device for diffusing solutions having a porous element impregnated with a solution having active agents, and a heating element to heat the porous element promoting the evaporation of the active agents contained in the solution absorbed by the porous element. The porous element includes the heating element in the form of a resistive material deposited on an outer surface portion of the porous element. Electrical contacts adapted for connection to an electrical power source are also provided on the layer of resistive material.

This application claims the benefit of filing priority under 35 U.S.C. §119 and 37 C.F.R. §1.78 of the co-pending Italian Patent Application No. MI2001A 000559 filed Mar. 16, 2001, for Device For Diffusion Of Solutions, Manufacturing Method For The Device And Electric Diffuser Using Said Device. All information disclosed in that prior pending foreign application is incorporated herein by reference.

### FIELD OF THE INVENTION

The present invention relates generally to a device for diffusing solutions, and in particular to solutions containing active agents, such as insecticide, deodorants, and disinfecting substances and the like. The present invention further relates to methods of manufacturing said device and to an electric diffuser using said device.

### BACKGROUND OF THE INVENTION

Systems for diffusing active agents into the air currently in use are based on heating a carrier holding a suspended or dissolved substance to be evaporated to a pre-selected temperature in accordance with the type of substance to be evaporated. Such systems generally comprise, in addition to elements for holding the carrier and the elements for enabling a connection to a power system, an element to hold the carrier and agent along with a heating element.

Some systems use single-dose systems, such as tablets composed of porous material impregnated with an active agent to be diffused, and some systems incorporate reusable elements which can convey liquid substances from a reservoir to an evaporation area. These last devices allow for multiple use cycles and, thus, diffusion of substances over a greater length of time.

The heating element may consist of a heat source, a metal layer or oxide resistance or piezoelectric element (PTC), and an layer of ceramic material that ensures electrical insulation. The heating element usually has a high capacity and thermal conduction and thereby allows the temperature to be made uniform with respect to the area where the product to be diffused is introduced.

Heating of the porous diffusion element containing the product takes place either by conduction, when the diffusion element is in direct contact with the heating body, or by convection. Initially, the air between the heating element and the diffusion element is heated and then the air subsequently heats the porous element. Depending upon the thermal characteristics of the materials making up the porous element currently in use, small variations in shape or position of the heating element can cause large variations in the temperature inside the porous diffusion elements and, therefore, can cause variations in the evaporation of the substance to be evaporated. Alterations in the structure of the porous diffusion element also can effect the evaporation rates. Since the amount of substance to be evaporated during the heating and evaporation process is relatively large (a heating element must interact with an element containing the substance to be evaporated), the power consumption of conventional devices is about 4 watts. Also, since the heating element may be in direct contact with portions of a supporting structure, the materials, normally plastic, forming said devices must have heat resistant properties, possibly in excess of their nominal thermal properties.

However, one limitation of current designs of electric diffusion devices is their low versatility to diffuse different types of substances. Once the supply voltage has been preset, most diffusion devices maintain a predetermined single operating temperature as determined by the electrical properties of the heat source. These operating conditions are necessarily optimised for diffusion of a specific substance. For different substances, for example products with varying degrees of volatility, the same working temperature conditions may lead to higher or lower evaporation rates and may prove unsuitable for different types of substances to be evaporated.

Therefore, what is needed is an electric diffuser that allows for efficient evaporation of varying types of substances over a range of operating conditions.

### SUMMARY OF THE INVENTION

It is the object of the present invention to overcome the drawbacks of prior diffusion devices by providing a diffusion device to evaporate active agents that is efficient, effective and versatile.

Another object of the present invention is to provide a manufacturing method for manufacturing a device for diffusion of active agents that is practical, economical and simple to carry out.

Another object of the present invention is to provide an electric diffuser with interchangeable refills that occupies little space and allows less waste of electrical power.

Another object of the present invention is to provide an electric diffuser with disposable refills that is practical and economical.

In summary, the primary elements of the herein disclosed electric diffuser comprise a container for holding a carrier substance with an active agent and a heating system. The heating system includes a porous diffusion element having a layer of electrically resistive material deposited over a selected surface area Electrodes are connected to an electrical power source, such as a house-hold electrical outlet, and connected to specific points on the deposited layer of material. A fixed voltage is then applied to the layer of resistive material causing an electric current to passes through it, thereby causing heat to be produce through a Joule effect. This causes the heating of the substrate of the porous element which results in the evaporation of the substance held in the container.

The device for diffusion of active agents according to the invention has various advantages. The device according to the invention is less bulky than prior art devices. In fact the resistive layer deposited on the surface of the porous element entirely replaces the external heating element of present electric diffusers. Therefore the instant diffuser device can be markedly reduced in size because the part relating to the external heating element is no longer present.

The invention also allows a considerable cost saving in the choice of materials for production of the container. In fact, given that the heat source is situated only on the porous element, the adjacent structure can be made of materials of less stringent material properties.

The invention allows a greater energy saving with respect to the similar devices. In fact, the deposited surface layer of resistive material optimises heat distribution on the diffusion element such that better thermal contact between the heating element and the substrate allows working temperatures to be reached very rapidly. Consequently, the improved thermal characteristics and optimised heat distribution make it possible to operate the invention at lower power levels (about 2 watts) than other similar devices used in the industry.

Further, the invention's design proves to be extremely versatile. In fact, the heating element portion can be deposited on porous elements of different shapes and sizes, and since the diffusion element having the integrated resistive heating element are interchangeable, it can be replaced as desired, and the invention can be used with a variety of substances having varying volatility characteristics by simply matching a particular diffusion element design with a particular carrier substance. This also allows for the usage of any electric diffusion device with varying substances over extended periods.

Other features and objects and advantages of the present invention will become apparent from a reading of the following description as well as a study of the appended drawings.

### BRIEF DESCRIPTION OF THE DRAWINGS

An apparatus for diffusion incorporating the features of the invention is depicted in the attached drawings which form a portion of the disclosure and wherein:

FIGS. 1-3 are perspective views illustrating the various stages of production of the diffusion element for diffusion of active agents in accordance with the disclosed invention;

FIG. 4 is a diagrammatic front view of an electric diffuser in accordance with the disclosed invention;

FIG. 5 is a top plan view of an element for coupling a refill for the diffuser of FIG. 4;

FIG. 6 is a side view of the coupling device of FIG. 5;

FIGS. 7-9 are, respectively, front, rear and side views of a refill for an electric diffuser in accordance with a second embodiment of the disclosed invention;

FIG. 10 is an exploded side view of an electric diffuser in accordance with a second embodiment of the disclosed invention;

FIG. 11 is a front view of the assembled electric diffuser of FIG. 10, in which a biasing element has been removed and the axis joining the poles of the plug is disposed parallel to a horizontal plane;

FIG. 12 is a view, like FIG. 11, wherein the biasing element has been added and the level of the liquid contained in the refill container is shown,

FIG. 13 is a front view, like FIG. 11, wherein the axis joining the poles of the plug is disposed at right angles to a horizontal plane;

FIG. 14 is a view, like FIG. 13, in which the biasing element has been added and the level of the liquid contained in the refill container is visible;

FIGS. 15 and 16 are, respectively, a front view and a rear view of a refill for an electric diffuser in accordance with a third embodiment of the invention;

FIG. 17 is a sectional view taken along the section line XVII-XVII of FIG. 15;

FIG. 18 is an exploded side view of an electric diffuser in accordance with a third embodiment of the invention;

FIGS. 19 and 20 are, respectively, a side view and a front view of the assembled electric diffuser of FIG. 18.

### DESCRIPTION OF THE PREFERRED EMBODIMENTS

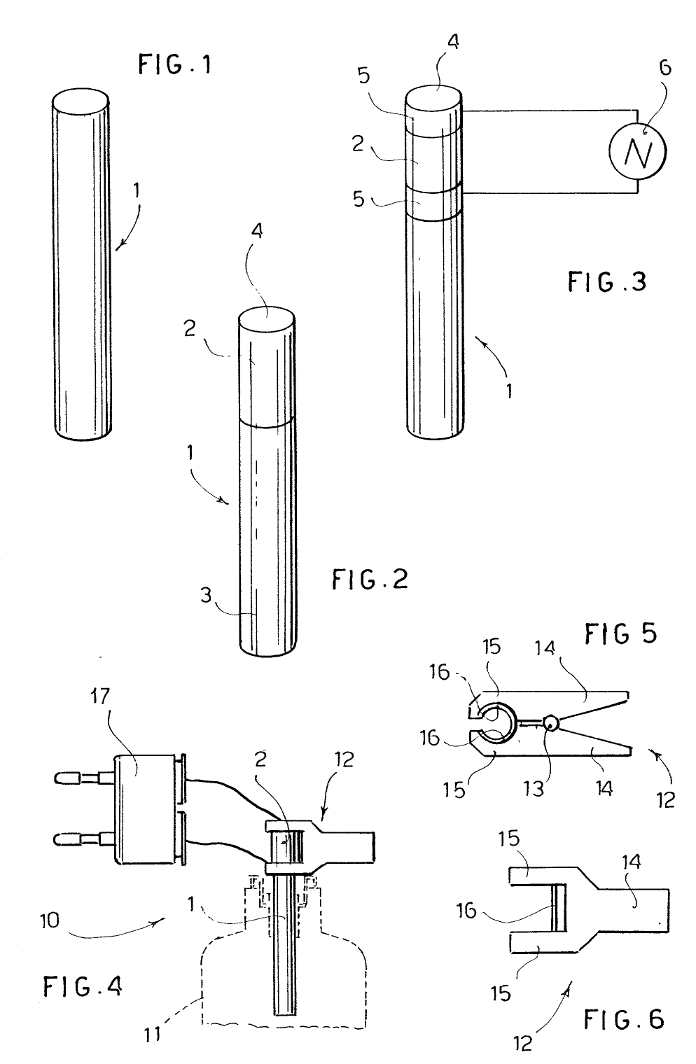

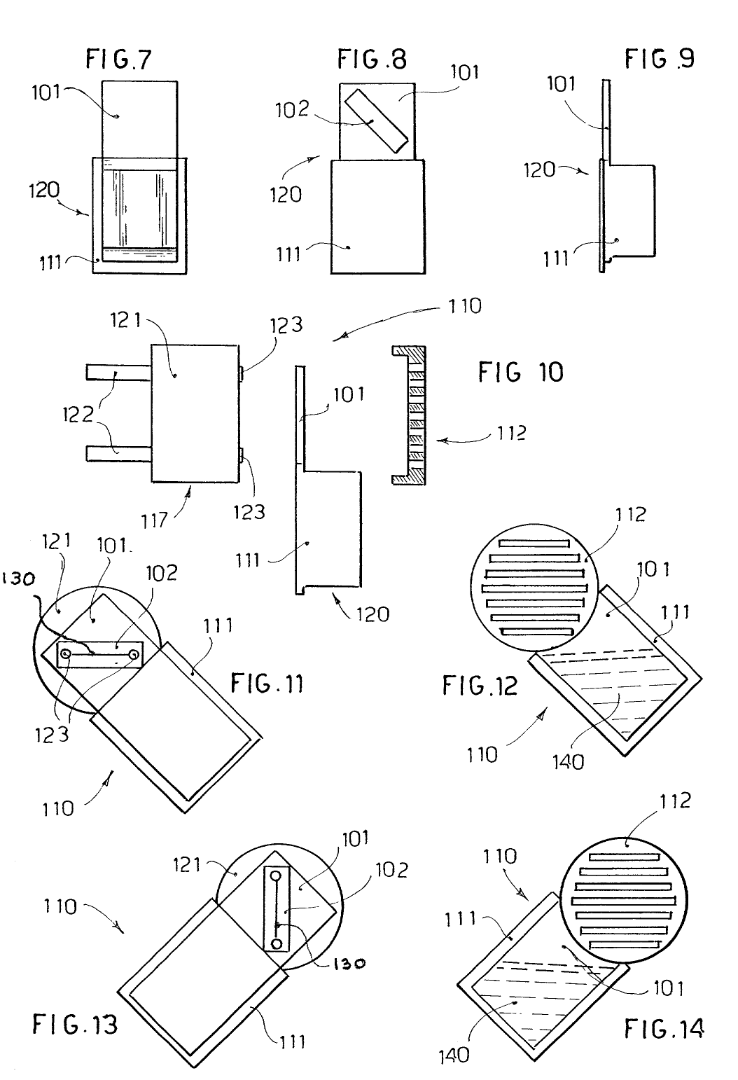

A device for diffusing active agents in accordance with the features of the invention is described with the aid of FIGS. 1-3. FIG. 1 shows a porous diffusion element having the form of a cylindrical column. The element 1 is made of a porous material able to absorb liquid solutions containing active agents. As shown in FIG. 2, a layer of electrically resistive material 2 having a known resistivity is deposited on the exterior surface of the porous element 1. The resistive material 2 is deposited at one end of the exterior surface of the porous diffusion element 1, leaving uncovered the other end 3 which is designed to be inserted into a container of liquid having one or more active agents.

Since the resistive material 2 is deposited only on the exterior surface of the porous element 1, the upper end 4 of the porous element 1 remains devoid of resistive material 2 and acts as an evaporating surface or opening to allow evaporation of the liquid solution containing the active agents absorbed by the porous element 1 from the other end 3.

As shown in FIG. 3, two electrical contacts 5 of conductive material are formed at the ends of the layer of resistive material 2. The electrical contacts 5 are connected by means of electrical cables to an electric power source 6 or to a plug to be inserted into an electrical socket outlet. Thus, a fixed voltage is applied to the resistive layer 2 and an electric current passes through it which produces heat through a Joule effect, causing heating of the substrate formed by the porous element 1 and causing evaporation of the liquid having active agents from the evaporation surface 4.

The resistive surface layer 2 may be produced by mixing two different inks, one a graphite based ink exhibiting properties of very low electrical resistivity and another ink exhibiting insulating properties. By mixing different concentration ratios of the two inks, the resistance of the deposited layer may be varied. To optimise the viscosity of the product and thus facilitate deposition thereof, a certain amount of diluting substance can be added to the ink mixture.

Deposition of this material can be performed by various methods, such as silkscreen printing, spray deposition, immersion deposition and other methods, depending upon the type and shape of the porous diffusion element that is to be treated.

The shape of the deposition area is not critical to the operation of the device and can be varied as needed to optimise the heating of the porous element 1 and facilitate a connection to the electrical contacts. However, the deposition area must not cover the entire porous diffusion element so that a portion can be exposed to the air unhindered to allow for release of the evaporated substance. Thus, for example, in the case of porous elements with a cylindrical shape, such as cylindrical wicks used in multiple dose diffusers, the electrically resistive layer is deposited on the outer surface of the upper portion of the cylinder that is, the end not inserted into the evaporative substance held in the reservoir, as shown in FIG. 2. In the case of planar porous diffusion elements, such as tablets and flat wicks, deposition is applied one of the two face surfaces of the element.

Once deposition has been performed, the system must undergo a thermal treatment that allows drying and polymerization of the resistive layer 2. The parameters of this treatment, temperature and time, depend on the type of inks that are used and the characteristics of the materials forming the porous elements. Normally these thermal elements are cured in conventional hot-air ovens. However, it is also possible to use infrared lamps that create radiant heating.

After the thermal treatment the porous heating element 1 is ready to be impregnated with the product to be evaporated and subsequently to be inserted and used in electric diffuser devices. One critical aspect of such devices is that a stable and safe electrical contact with the resistive layer 2 must be maintained so that the dimensions of the resistive layer and thus the resistivity do not vary, even after multiple insertions of new diffuser refills are made. This is important so that the performance of device remains constant.

An example of such a diffuser is shown in FIG. 4. The electric diffuser 10 uses a cylindrical porous heating element 1 inserted into a replaceable container 11 that holds a solution containing active agents. The electric diffuser 10 provides a coupling device 12 to allow for coupling and uncoupling of the container 11 and the diffuser heating element, which is in electrical contact with the resistive layer 2.

As shown in FIGS. 5 and 6, the device 12 comprises two levers 14, defining two end jaws 15, coupled and biased together by means of a torsion spring 13. By closing the outer levers 14 the spring 13 is compressed and the jaws 15 of the device 12 open, thus allowing either the insertion of a new container 11 or the release of the spent container. Upon releasing the outer levers 14, if a container is present the jaws 15 always grip the upper part of the porous element 1 at the same location, that is to say the part on which the resistive layer 2 is deposited. Metal blades 16, electrically connected with an electric power plug 17 are positioned at fixed points on the ends of the coupling device 12 where contact with the porous element occurs. Therefore, the metal blades 16 of the coupling device 12 provide a stable electrical contact with the surface resistive layer 2 and an electrical connection to power plug 17 and a power source.

As is known in the art, coupling device 12 can also include additional electrical circuitry so that the electric diffuser 10 may be turned on and off, possibly at timed intervals, without forcing physical disconnection of the device from an electrical outlet.

The electric diffuser 10 has been designed to include the coupling device 12 for cylindrical porous elements such as the one shown. However, the shown electric diffuser 10 is not of the disposable type, but can used with a large number of container refills. Consequently, the coupling device 12 is designed for multiple coupling/uncoupling actuations of the container refill and is structured to ensure consistent positioning of the container refill and the porous diffuser element 1 to provide sufficient electrical contact between the resistive film 2 deposited on the porous element 1 and coupling device 12.

A second and a third embodiment of electric diffusers of the disposable type using porous elements with a deposited resistive film according to the invention will be illustrated below.

A second embodiment of the electric diffuser in accordance with features of the disclosed invention are now described with the aid of FIGS. 7-14. FIGS. 7-9 show a container refill denoted as a whole by reference numeral 120. For the sake of simplicity, the term “refill” is used even if it is destined to be incorporated in an electric diffuser of the disposable type. The refill 120 comprises a container 111 wherein a solution with active agents is contained, a plate-shaped porous element 101 partially inserted in the container 111, and a heating element 102 deposited on the porous element 101.

The porous element 101 consists of a material such as cardboard, plastic or other porous material that allows the liquid to be conveyed by capillary action from the container 111 to a targeted evaporation area outside the container. The heating element 102, in the form of a thin film or electrically resistive material, is deposited on the porous element 101, as already described. The heating element is in the form of a strip inclined 45° with respect to the axis of the porous element.

The container 111 can consist of any transparent plastic material to allow direct visual inspection of the amount of liquid present therein. The container 111 can be comprised of multiple elements, but must be made so as to provide a completely tight seal in the region of the porous element 101 to prevent the liquid from escaping or the porous element from being displaced relative to the container 111, regardless of the orientation in which the refill is positioned.

FIG. 10 is an exploded view of an electric diffuser 110 according to a second embodiment. The electric diffuser 110 comprises the refill 120, an electric plug 117 and a biasing element 112. The electric plug 117 includes a body 121, two poles 122 for connection to an electrical outlet, and two electrical contacts 123 adapted to come into contact with the resistive element 102 deposited on the porous diffusion element 101. The biasing element 112 is responsible for biasing the plug 117 so that the electrical contacts 123 press against the resistive element 102. The biasing element is made of insulating material to ensure adequate electrical insulation of the biased parts and has a grille or slotted configuration to allow substances evaporated during operation of the electric diffuser 110 to escape into the environment. Since the electric diffuser 110 would typically be disposable, the individual elements are assembled in such a way as to prevent disassembly.

FIGS. 11 and 13 show the electric diffuser 110 inserted into electrical outlets having contact sockets oriented horizontally (FIG. 11) and vertically (FIG. 13). Resistive film 102 has its length oriented at a 45° angle relative to a parallel axis extending along the length of the refill 120 as shown, and electrical contacts 123 connect with the resistive film 102 along a axis 130 parallel to the length of the film. This offset orientation allows for continuous contact of the porous element 101 with the liquid 140 inside the container 111 regardless of the orientation of the electrical socket and regardless of the level of liquid remaining in the container 111, as shown in FIGS. 12 and 14. This also obviates the need to provide the electric diffuser 110 with a rotating plug.

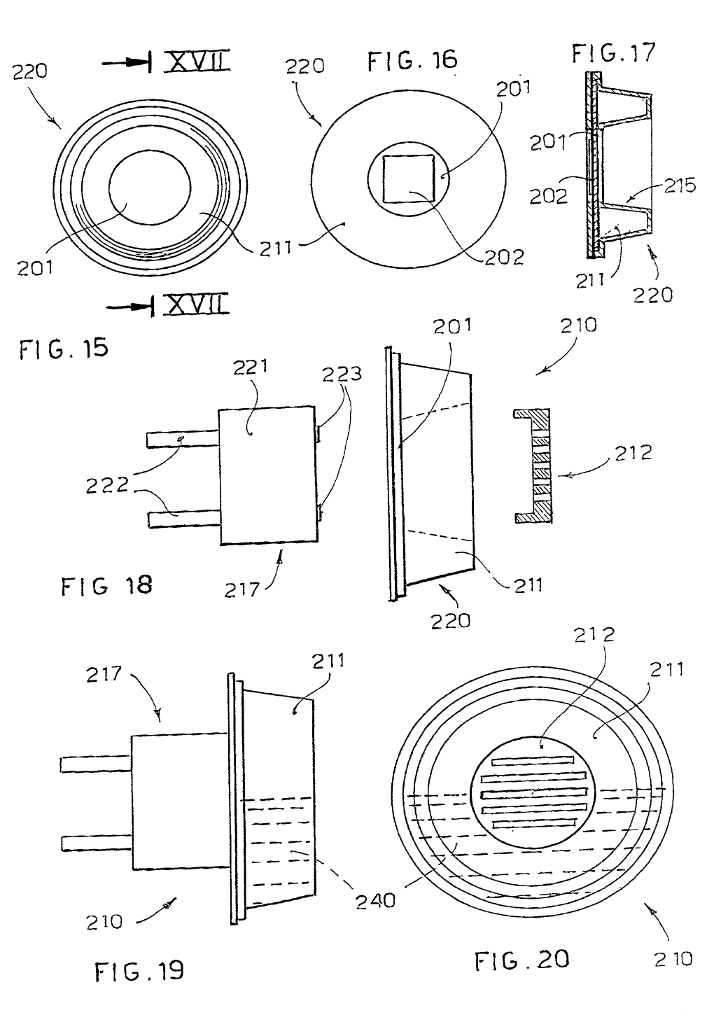

A third embodiment of the electric diffuser according to the invention is described with the aid of FIGS. 15-20. FIGS. 15-17 show a refill denoted as a whole with reference numeral 220. The refill 220 is formed in the shape of a toroid or doughnut defining a container 211 and having an aperture 215 through its center. The container 211 holds the solution to be evaporated with its active agents. A porous diffusion element 201 in the shape of a disc is inserted into the container 211 so that a peripheral portion of the of the disc shaped porous element 201 is positioned inside the container 211, thereby being in constant contact with any liquid held inside the container 211, and a central portion of the porous element extends over the central aperture 215, thereby leaving exposed to the air the central portion of the porous element. A heating element 202 is deposited on the rear side of the central portion of the porous element 201 not covered by the container 211.

The porous element 201 is made of material such as cardboard, plastic or other porous material that allows the liquid to be conveyed by capillary action from the container 211 to the central area of the porous element 201 which represents the evaporating area.

The container 211 can be formed of any transparent plastic material to allow visual inspection any liquid present therein. The container 211 may be composed of multiple sections, but must be formed to ensure a tight seal around the porous element and especially at the contact surface areas along the inner periphery of the aperture 215 of the container 211 of the porous element to prevent the liquid contained therein from leaking or the porous element 201 from being removed, independently of the orientation in which the refill 220 is positioned.

FIG. 18 is an exploded view of an electric diffuser 210 in accordance with the features of the third embodiment. The electric diffuser 210 comprises the refill 220, an electric plug 217, and a biasing element 212 similar to one already described in the second embodiment. The electric plug 217 comprises a body 221, two pins 222 for connection to an electrical outlet and two electrical contacts 223 adapted to make electrical contact with the resistive element 202 deposited in the central part of the rear surface of the porous element 201.

As in the second embodiment, the biasing element 212 is responsible for biasing the plug 217 so that the electrical contacts 223 press onto the resistive element 202. The biasing element is made of insulating material to ensure adequate electrical insulation to the electrically conductive portions of the invention and has a grill-type or slotted configuration to allow the evaporated substances to escape into the air during operation of the electric diffuser 210. Assembly of the individual elements of the electric diffuser 210 are done in such a way as to prevent disassembly.

FIGS. 19 and 20 show the assembled third disclosed diffuser embodiment 210 with a transparent container 211 to allow inspection of the level of the liquid 240 contained therein. The symmetrical design of the electric diffuser 210 allows it to be used regardless of the orientation of a socket of a wall-mounted electrical outlet, thereby obviating the need for a rotating plug. As may be seen, the porous element 201 always remains in contact with the liquid 240 inside the container 211 until all of the liquid has been evaporated.

While the invention has been shown in various forms, it will be obvious to those skilled in the art that it is not so limited but is susceptible of various changes and modifications without departing from the spirit thereof.

Having set forth the nature of the present invention, what is claimed is:

1. A device for diffusion of solutions containing active agents, comprising:
a porous diffusion element impregnated with said solution;
a heating element to heat said porous element promoting evaporation of said active agents contained in said solution; and,
wherein said heating element comprises a layer of resistive material deposited upon a portion of said porous diffusion element.

2. A device as recited in claim 1, wherein said layer of resistive material is adapted to receive electrical contacts in electrical communication with an electric power source.

3. A device as recited in claim 1 or 2, wherein said layer of resistive material comprises a mixture of two inks, one said ink being graphite-based having low electrical resistivity and said other ink having insulating properties.

4. A device as recited in claim 1 or 2, wherein said porous diffusion element includes an area devoid of said layer of resistive material to facilitate evaporation of said active agents.

5. A device as recited in claim 3, wherein said porous diffusion element includes an area devoid of said layer of resistive material to facilitate evaporation of said active agents.

6. A device as recited in claim 1 or 2, wherein said porous element comprises a cylindrical shape and wherein said layer of resistive material is deposited toward one end of said porous element.

7. A device as recited in claim 3, wherein said porous element comprises a cylindrical shape and wherein said layer of resistive material is deposited toward one end of said porous element.

8. A device as recited in claim 4, wherein said porous element comprises a cylindrical shape and wherein said layer of resistive material is deposited toward one end of said porous element.

9. A device as recited in any one of the claims 1-2, wherein said porous element comprises a plate shape and said layer of resistive material is deposited on a portion of only one face of said porous element.

10. A method for manufacturing an element for diffusing solutions with active agents comprising the following steps:
producing a porous element capable of absorbing a solution holding active agents;
depositing a layer of electrically resistive material over a portion of said porous element; and,
applying heat to said porous element to allow drying and polymerization of said layer of resistive material.

11. A method for manufacturing as recited in claim 10, wherein said deposition step comprises the step of silkscreen printing, spray deposition, or immersion deposition.

12. A method for manufacturing as recited claim 10 or 11, wherein said step of applying heat comprises treatment in a conventional hot-air oven or radiant heating by means of infrared lamps.

13. An electric diffuser for diffusion of solutions containing active agents, comprising:
a refill having a container holding a solution with active agents and a porous diffusion element impregnated with said solution, wherein a surface portion of said diffusion element includes a layer of resistive material; and
a coupling device for replacement of said refill and for electrical contact with said layer of resistive material, wherein said coupling device is in electrical contact with an electrical power plug.

14. An electric diffuser as recited in claim 13, wherein said coupling device further comprises two levers biased against one another with a torsion spring, and wherein said two levers are adapted to form two jaws for clamping said porous element on said layer of resistive material.

15. An electric diffuser as recited in claim 13 or 14, wherein said coupling device includes metal blades for contacting said layer of resistive material and wherein said metal blades are electrically connected to an electric power plug.

16. An electric diffuser for diffusion of solutions containing active agents, comprising:
a refill comprising:
a container containing a solution with active agents,
a porous element impregnated with said active agents, and
a layer of resistive material deposited on a surface portion of said porous element;
an electrical plug for connection to an electrical outlet, said plug including electrical contacts adapted to contact said resistive layer deposited on said porous element; and
a coupling device connecting said electrical plug to said refill, said coupling device adapted to force said electrical contacts of said electrical plug into electrical contact with said resistive layer deposited on said porous element.

17. An electric diffuser as recited in claim 16, wherein said porous element is plate shaped and partially inserted into said container, and wherein said resistive layer comprises a strip disposed on a portion of said porous element outside of said container, and wherein said strip includes an axis along its greatest length and said porous element includes an axis along its greatest length, said strip axis forming an angle of 45° relative to said porous element axis.

18. An electric diffuser as recited in claim 16, wherein said porous element is disc shaped and said container is doughnut shaped, said container having an inner circumference defining a center aperture, and wherein said porous element is integrated with said container such that a portion of said porous element is exposed to ambient air through said center aperture on one side and wherein said layer of resistive material is disposed on the other side of said porous element within a central portion of said porous element.

19. An electric diffuser as recited in any one of claims 16-18, wherein said coupling device is comprised of insulating material to ensure electrical insulation and includes holes or slots to allow for diffusion of said active agents.
